(19) United States
(12) Patent Application Publication
(10) Pub. No.: US 2025/0359813 A1
(43) Pub. Date: Nov. 27, 2025

(54) SYSTEM AND METHOD OF DETECTING ELECTROPHYSIOLOGICAL EVENTS IN A SUBJECT

(71) Applicants: RAMOT AT TEL-AVIV UNIVERSITY LTD., Tel Aviv (IL); ICHILOV TECH LTD., Tel-Aviv (IL)

(72) Inventors: Yuval NIR, Tel Aviv (IL); Rotem FALACH, Tel Aviv (IL); Firas FAHOUM, Ramat Gan (IL)

(21) Appl. No.: 19/293,820

(22) Filed: Aug. 7, 2025

Related U.S. Application Data
(63) Continuation of application No. PCT/IL2024/050053, filed on Jan. 15, 2024.
(60) Provisional application No. 63/445,318, filed on Feb. 14, 2023.

(51) Int. Cl. A61B 5/00; A61B 5/31; A61B 5/37

(52) U.S. Cl. CPC A61B 5/4094; A61B 5/31; A61B 5/37; A61B 5/7267

(57) ABSTRACT

A system and method of detecting Electrophysiological events such as Interictal Epileptiform Discharge (IED) events, or other pathological and physiological electrophysiological events, in a human subject by at least one processor may include, for example: placing at least one first electroencephalogram (EEG) electrode over a zygomatic bone or a maxilla of the subject, directly below the subject's orbit in the subject's inferior direction; receiving a first EEG signal from the at least one first EEG electrode; processing the first EEG signal, to obtain one or more first EEG data elements; and inferring at least one machine-learning (ML) based model on the one or more first EEG data elements, to predict occurrence of at least one IED event in the subject.

### CROSS REFERENCE TO RELATED APPLICATIONS

This application is a ByPass Continuation of PCT International Application No. PCT/IL2024/050053, having international filing date of Jan. 15, 2024, which claims the benefit of priority to U.S. Provisional Patent Application No. 63/445,318, filed Feb. 14, 2023, entitled “SYSTEM AND METHOD OF DETECTING ELECTROPHYSIOLOGICAL EVENTS IN A SUBJECT”, the contents of which are all incorporated herein by reference in their entirety.

### FIELD OF THE INVENTION

The present invention relates generally to assistive diagnosis technology. More specifically, the present invention relates to systems and methods of detecting Electrophysiological (EP) events such as Interictal Epileptiform Discharge (IED) events in a subject by at least one processor.

### BACKGROUND OF THE INVENTION

Epilepsy is one of the most common neurological conditions, affecting over 70 million people worldwide. Pathological Electrophysiological (EP) events of electrical discharges occur spontaneously between seizures, and may include for example, Electroencephalogram (EEG) slowing, High Frequency Oscillations (HFOs), and Interictal Epileptiform Discharges (IEDs), which are fast and sharp discharges, which can be accompanied by a slow wave activity, commonly referred to as “spike-wave complex”.

EP events such as IEDs have clinical significance, and are associated with seizures as well as with long-term cognitive decline. IEDs are typically most frequent during NREM sleep, potentially disrupting memory consolidation. IEDs may also occur in a wide array of neurological conditions beyond epilepsy, such as dementia, autism, following Traumatic Brain Injury (TBI), stroke, encephalitis, and the like.

Detection of EP events such as IED using scalp EEG is possible mostly when pathological activity is apparent in the lateral cortical regions, but is highly challenging, and often impossible, when the EP events occur in the hippocampus and surrounding Medial Temporal Lobe (MTL) regions.

### SUMMARY OF THE INVENTION

Embodiments of the invention include a machine learning (ML) based platform for detecting MTL epileptic activity, or other pathological (e.g., epileptic) or physiological sleep electrophysiological events such as High Frequency Oscillations (HFOs), ripples, slow waves, or sleep spindles, by at least one processor, in a non-invasive manner, e.g., based on zygomatic EEG data.

The inventors used a unique opportunity to develop ML models using data simultaneously recorded from the MTL, and from a few facial EEG electrodes in pharmaco-resistant epilepsy patients implanted with depth electrodes for clinical evaluation.

Embodiments of the invention include a first ML model for detection EP events such as IEDs in individual MTL depth electrode channels, trained with a dataset of manually tagged events by an expert neurologist.

After preprocessing and segmentation to short time windows, multiple spectral, time-domain, and statistical features were extracted for each data segment. Next, the inventors trained models, focusing on decision tree-based algorithms (Random Forest, Light Gradient Boost Machine), detecting all EP events (e.g., IEDs) occurring overnight. Performance was evaluated with metrics of precision and recall.

Second, the inventors used the first model's output of MTL EP events (e.g., IEDs) as the ground truth input to train a second model with features extracted from non-invasive facial EEG channels only. Expectedly, the sensitivity of MTL EP events detected non-invasively was lower than that originating from intracranial measurements but, importantly, the detection precision for the subset of detected events remained significantly high (>75%).

Our work establishes that reliable detection of a minority of MTL EP events (e.g., IEDs) is possible in non-invasive EEG data, opening several new avenues to improve diagnosis, prognosis, drug treatment and risk-stratification in diverse neurological conditions associated with interictal activity during sleep including but not limited to epilepsy such as TBI, Alzheimer's disease and other forms of dementia.

Moreover, the same method can be used to non-invasively detect markers of MTL activity including pathological interictal/seizure activities, EEG slowing, or even healthy hippocampal electrophysiological activity.

Embodiments of the invention may include a method of detecting Electrophysiological (EP) events in a human subject by at least one processor.

Embodiments of the method may include placing at least one first electroencephalogram (EEG) electrode over a zygomatic bone or a maxilla of the subject, directly below the subject's orbit in the subject's inferior direction. The at least one processor may receive a first EEG signal from the at least one first EEG electrode, and process the first EEG signal, to obtain one or more first EEG data elements, representing electrical activity in the subject's medial temporal lobe (MTL).

The at least one processor may subsequently infer at least one machine-learning (ML) based model on the one or more first EEG data elements, to predict occurrence of at least one EP event in the subject, such as an Interictal Epileptic Discharge (IED) event.

The EP events may include, for example, IED events, pathological events, physiological sleep electrophysiological events, High Frequency Oscillation (HFO) events, ripple events, slow wave events, and spindle events.

According to some embodiments, the at least one ML model may include a prediction model. The ML-based prediction model may for example, include, or be implemented as a decision-tree model or a gradient-boost ML model. The prediction model may be pretrained to predict the occurrence of EP events based on the one or more first EEG data elements.

Additionally, or alternatively, the at least one ML model may also include a classification model, pretrained to automatically produce at least one annotation that may indicate occurrence of an EP event (e.g., IED) in the subject at a timeframe that corresponds to the first EEG signal.

According to some embodiments, the at least one processor may use the automatically produced annotation as supervisory data, to train the prediction model to predict the occurrence of EP events based on the one or more first EEG data elements.

For example, the at least one processor may receive a second EEG signal, originating from at least one second, intracranial EEG electrode, and concurrent with the first EEG signal. The at least one processor may also receive a label data element, indicating occurrence of an EP event (e.g., IED) in the subject, and may use the label data element as supervisory information to train the classification model, to automatically produce the at least one annotation based on the second EEG signal.

Additionally, or alternatively, the at least one processor may produce a diagnosis of a medical condition of the subject based on said predicted occurrence of EP events. The medical condition may include, for example epilepsy, autism, Alzheimer's disease, neurodegeneration, dementia, Traumatic Brain Injury (TBI), Post Traumatic Stress Disorder (PTSD), abnormal brain activity following neurosurgery, existence of brain tumors, anxiety, depression, psychosis, chronic headache or migraine, and stroke.

Embodiments of the invention may include a system for detecting EP events in a human subject. Embodiments of the system may include a non-transitory memory device, where modules of instruction code are stored, and at least one processor associated with the memory device, and configured to execute the modules of instruction code.

Upon execution of said modules of instruction code, the at least one processor may be configured to receive a first EEG signal from at least one first EEG electrode, wherein said at least one first EEG electrode is placed over a zygomatic bone or a maxilla of the subject, directly below the subject's orbit in the subject's inferior direction; process the first EEG signal, to obtain one or more first EEG data elements; and infer at least one ML based model on the one or more first EEG data elements, to predict occurrence of an EP event (e.g., an IED event) in the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included in a system for detecting EP events in a human subject, according to some embodiments;

FIG. 2 is a block diagram, depicting a system for detecting EP events in a human subject, according to some embodiments;

FIG. 3 is a flow diagram, depicting a method of detecting EP events in a human subject, according to some embodiments;

FIG. 4 is a flow diagram, depicting another method of detecting EP events in a human subject, according to some embodiments;

FIGS. 5A-5D show experimental results of detecting EP events in a human subject, by a depth model, according to some embodiments;

FIGS. 6A-6D show experimental results of detecting EP events in a human subject, by a basic version of a zygomatic EEG (zEEG) model, according to some embodiments; and

FIGS. 7A-7C show experimental results showing selection of EEG electrodes by an advanced version of a zEEG model, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Embodiments of the present invention may include a method and a system for non-invasive detection of pathological and/or physiological EP activity such as IEDs, occurring in deep brain regions such as the medial temporal lobe (MTL). Embodiments may include a machine learning based tool trained on unique data recorded simultaneously intracranially and non-invasively with EEG and/or face electrodes. Embodiments of the invention may allow non-invasive, reliable detection of MTL EP events (e.g., IEDs), as well as other pathological and/or physiological activities, and may be applicable in a diverse range of clinical applications, such as neurodegeneration, autism, TBI, and possibly monitoring of healthy hippocampal electrophysiological activity.

As known in the art, IEDs are brief paroxysmal electrographic events observed between spontaneous recurrent seizures in epilepsy patients. IEDs (i) have a duration of 70-200 ms (for a sharp wave) or 20-70 ms (for a spike), (ii) entail an abrupt change in polarity, (iii) have a restricted physiological spatial field, and (iv) are most prevalent in non-rapid eye movement (NREM) sleep. IEDs occurring in the MTL during sleep may impair memory by affecting hippocampal-cortical coupling, and their reliable detection has clinical value in epilepsy and other neurological conditions.

The inventors set out to develop and validate automatic detection of EP events such as IEDs with a machine learning approach in intracranial EEG (iEEG) and in non-invasive facial (zygomatic) EEG.

During the development process, a cohort of drug-resistant mesial temporal lobe epilepsy (MTLE) patients underwent clinical pre-surgical evaluation and were implanted with intracranial depth electrodes in the MTL. Overnight iEEG signals were recorded, and referenced to a central scalp electrode, sampled at 2 KHz, and bandpass filtered between 0.1 Hz and 500 Hz. Sleep was scored using established guidelines of the American Academy of Sleep Medicine.

The inventors focused on three channels per hemisphere: the anterior hippocampus, referenced to a midline central electrode (commonly referred to as a Cz electrode), the anterior hippocampus bipolar referenced to adjacent electrode (5 mm more laterally), and the amygdala, referenced to Cz.

The recorded signals were preprocessed, including segmentation of the signal to 250 ms intervals and extraction of signal features for the current and the previous interval, such as spectral power in specific frequency bands and statistical features such as variance and skewness.

The intervals were randomly split into train and test subsets, and were used to train two ML models: a random forest model, and a gradient-boost classifier. (e.g., LightGBM, XGBoost).

The first task aimed at detecting EP events (e.g., IEDs) in iEEG. To this end, the inventors used a dataset that contained EEG recordings during non-REM sleep. IEDs were visually scored by an expert neurologist.

The second task aimed at detecting EP events (e.g., IEDs) in a limited number of scalp EEG (Fz, Cz, Pz) and other facial electrodes such as Zygomatic electrodes. To this end, the inventors used the results from the first model on the entire overnight dataset (e.g., overall: over 15,000 events). This dataset contained a plurality of detected EP events (e.g., overall: 40 IED events over 6 hours), as tagged by the random forest classifier in non-invasive data.

For each task and algorithm, the inventors assessed the test results using standard metrics of precision (number of positive class predictions that indeed belong to the positive class) and recall (also known as sensitivity; number of positive class predictions out of all positive examples in the dataset).

Results: Results of the first task (automatic detection in intracranial data) were assessed by comparing model outputs to manual annotation by expert neurologists. The inventors obtained with random forest classifier: precision (e.g., 92%) and recall (e.g., 66%), and with the gradient-boost classifier: precision (e.g., 88%) and recall (e.g., 74%).

Results of the second task (automatic detection in scalp EEG/Zygomatic electrodes) were assessed by comparing model outputs to the automatic intracranial results. The inventors obtained with random forest classifier: precision (e.g., 77%) and recall (e.g., 3%), and with the gradient-boost classifier: precision (e.g., 67%) and recall (e.g., 4%). In other words, embodiments of the invention may facilitate automatic detection of presence of a subset of EP events (e.g., IEDs) in the MTL, with acceptable (>75%) precision non-invasively.

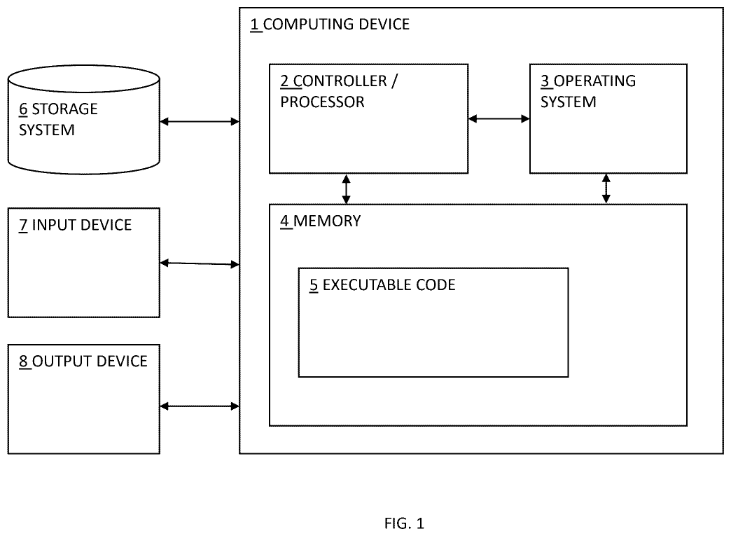

Reference is now made to FIG. 1, which is a block diagram depicting a computing device, which may be included within an embodiment of a system for detecting EP events (e.g., IEDs), according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may detect EP events (e.g., IEDs) as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to recording of EEG signals may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

The term neural network (NN) or artificial neural network (ANN), e.g., a neural network implementing a machine learning (ML) or artificial intelligence (AI) function, may be used herein to refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. At least one processor (e.g., processor 2 of FIG. 1) such as one or more CPUs or graphics processing units (GPUs), or a dedicated hardware device may perform the relevant calculations.

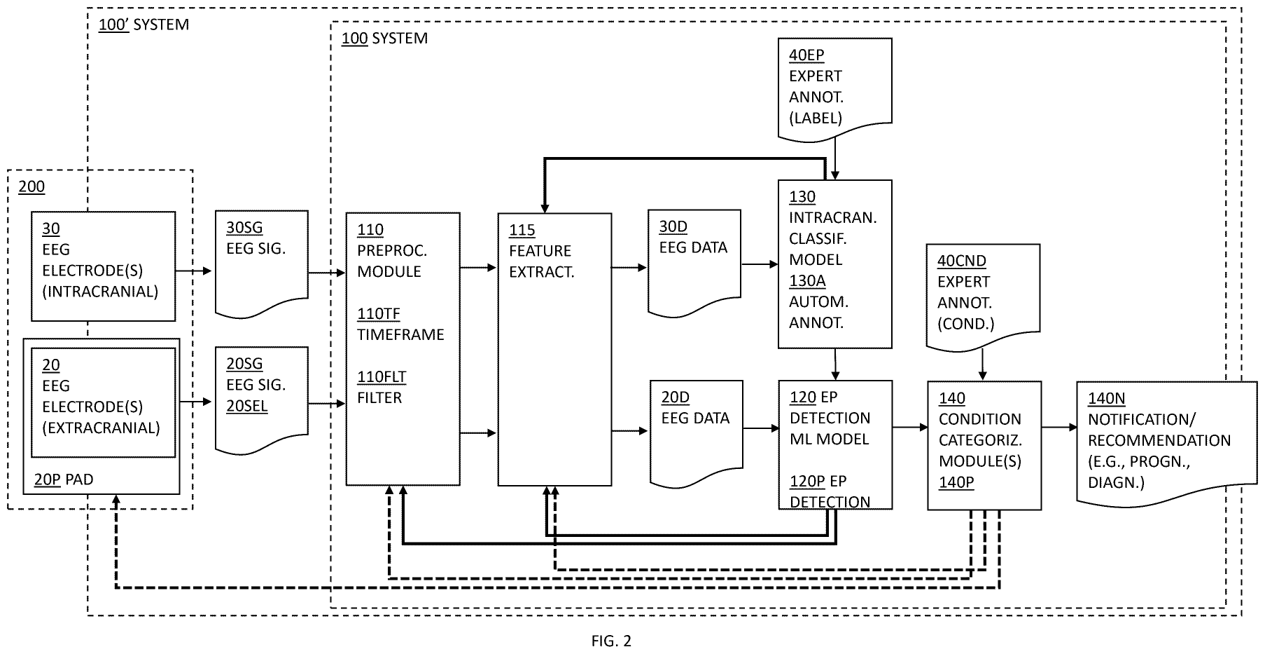

Reference is now made to FIG. 2, which depicts a system 100 for detecting EP events (e.g., IEDs) by at least one processor, according to some embodiments of the invention.

According to some embodiments of the invention, system 100 may be implemented as a software module, a hardware module, or any combination thereof. For example, system may be or may include a computing device such as element 1 of FIG. 1, and may be adapted to execute one or more modules of executable code (e.g., element 5 of FIG. 1) to detect EP events (e.g., IEDs), as further described herein. Arrows of system 100 may represent flow of one or more data elements to and from system 100 and/or among modules or elements of system 100. Some arrows have been omitted in FIG. 2 for the purpose of clarity.

As shown in FIG. 2, a system 100 for detecting EP events (e.g., IEDs) may be associated with, or connected e.g., via wired or wireless connection to at least one EEG electrode (e.g., 20\30), adapted to produce EEG signals (e.g., 20SG\30SG respectively) representing electrical activity within a subject's head.

Additionally, or alternatively, a system for detecting EP events (e.g., IEDs) may include the at least one EEG electrode (e.g., 20\30). In such embodiments the system 100 for detecting EP events is denoted as system 100′.

System 100′ may include, or may be associated with an EEG device 200, coupled with one or more EEG electrodes 20/30; a non-transitory memory device (e.g., memory device 4 of FIG. 1), wherein modules of instruction code are stored; and at least one processor (e.g., processor 2 of FIG. 1) associated with the memory device, and configured to execute the modules of instruction code.

As elaborated herein, upon execution of the modules of instruction code, the at least one processor 2 may obtain, via EEG device 200 at least one extracranial signal 20SG from at least one respective extracranial EEG electrode 20, which may be placed at a predetermined position over a zygomatic bone or a maxilla of a subject. The at least one processor 2 may process the at least one extracranial signal 20SG, to obtain one or more extracranial data elements 20D. The at least one processor 2 may then infer at least one ML based, EP detection model 120 on the one or more extracranial data elements 20D, to detect occurrence of at least one EP event 120P in the extracranial signal.

According to some embodiments, at least one extracranial EEG electrode 20 may be a non-invasive electrode, which may be placed on a face of the subject. For example, EEG electrode 20 may be placed over a zygomatic bone, or a maxilla of the subject, directly below the subject's orbit in the subject's inferior direction (e.g., directly beneath the subject's eye).

As shown in FIG. 2, system 100 may include a preprocessing module 110, configured to obtain at least one extracranial EEG signal 20SG from the at least one respective extracranial EEG electrode 20. The at least one extracranial EEG electrode 20 may be placed at a predetermined position over a zygomatic bone or a maxilla of the subject.

Selection of this location may provide several benefits for identifying EP events in a subject, and subsequent diagnosis of the subject's condition. For example, previous works have shown attempts to detect IED pulses from a multitude of extracranial EEG signals originating from a respective multitude of EEG electrodes in an EEG cap, placed on the scalp. Such works relied upon the (erroneous) inherent assumption that such signals may best represent electrical activity in a patient's medial temporal lobe (MTL). The inventors have experimentally shown that carefully placed, few (e.g., single) extracranial EEG electrodes 20 on the zygomatic bone may better reflect the electrical activity in the MTL, and may therefore be preferable to identifying EP events, and determining the patient's condition. It may also be appreciated that configurations of embodiments of the invention (e.g., using single, extracranial EEG electrodes 20 on the zygomatic bone) may (a) be less cumbersome than using an EEG cap, and (b) require fewer computing resources for analyzing the multitude of EEG signals produced by scalp EEG caps.

In another example, as known in the art, sleep clinics commonly use EEG measurements from electrodes placed beneath a patient's orbit, to efficiently analyze their sleep patterns. Configurations of embodiments of the invention (e.g., using single, extracranial EEG electrodes on the zygomatic bone) may thereby exploit this practice, to (a) allow patient screening via sleep clinics, and (b) correlate identified EP events with sleeping patterns, to fine tune determination of a patient's condition.

According to some embodiments, preprocessing module 110 may be configured to process extracranial signal 20SG, to obtain one or more extracranial EEG data elements 20D. Extracranial data elements 20D may represent electrical activity in the subject's MTL.

For example, preprocessing module 110 may include electrical circuitry configured to sample extracranial signal 20SG at a predefined sampling rate (e.g., 2 Kilo Hertz (KHz)), and apply a band-pass filter (e.g., in the range of 0.1 Hz and 500 Hz) on the sampled extracranial signal 20SG. Preprocessing module 110 may exclude noisy temporal intervals based on amplitude threshold or identification of other deviations from typical signal statistics, and normalize extracranial signal 20SG, e.g. to force a standard dynamic range of amplitude values.

Additionally, or alternatively, preprocessing module 110 may include electrical circuitry configured to apply analog to digital (A2D) conversion of the filtered extracranial signal 20SG, to produce an extracranial data element 20D that represents a digital, sampled and filtered version of extracranial signal 20SG.

Additionally, or alternatively, preprocessing module 110 may include a feature extraction module 115, configured to extract one or more extracranial data elements 20D that may represent statistical features of electrical brain activity in the subject's MTL such as standard deviation, skewness, kurtosis, entropy and complexity measures, and power in specific frequency bands (e.g. alpha/beta/gamma) and their ratios.

Additionally, or alternatively, preprocessing module 110 may produce the one or more extracranial data elements 20D such that each extracranial data element 20D may be associated with, or correspond to a predetermined timestamp or timeframe 110TF.

As shown in FIG. 2, system 100 may include a machine-learning (ML) based model, denoted herein as EP detection model 120. As elaborated herein, during a training stage, system 100 may train EP detection model 120 to receive one or more extracranial data elements 20D, and predict occurrence of at least one EP event in the subject, based on the received one or more extracranial data elements 20D.

According to some embodiment, the at least one ML based EP detection model 120 may include an ML model or architecture such as a decision-tree based model. For example, EP detection model 120 may be, or may include a random forest model, a Light Gradient Boost Machine (LGBM) model, a gradient-boost ML model, and an Extreme Gradient Boost (XGB) model, a gradient-boost ML model, or any combination thereof.

It may be appreciated that selection of EP detection model 120 as a decision-tree based model may provide a number of benefits for predicting occurrence of EP events (e.g., IEDs), in timeframes 110TF, based on the one or more extracranial data elements 20D.

For example, decision-tree based models may be preferable (e.g., in relation to deep-learning architectures) to classify incoming data (in this case-identify occurrence of EP events), based on scarcely annotated training datasets (as is the case for expert-labeled EEG signals).

In another example, decision-tree based models may be preferable (e.g., in relation to deep-learning architectures) to interpret, or explain the effect of specific nodes or features within the model on a subsequent decision (e.g., a condition of a patient). As elaborated herein, such enhanced explainability (as commonly referred to in the art) may facilitate a feedback mechanism, as depicted by the thick arrows of FIG. 2. This feedback mechanism may allow system 100 to select specific EEG electrodes, specific EEG signals 20SG, and/or parameters of preprocessing module 110, to fine tune the processing of EEG signals 20SG into extracranial data elements 20D, and improve identification of occurrence of EP events.

According to some embodiments, system 100 may (e.g., during an inference stage) provide one or more extracranial data elements 20D, obtained from EEG signals 20SG of non-invasive, extracranial electrodes 20 as input for ML-based EP detection model 120.

As elaborated herein, EP detection model may be pretrained to detect the occurrence of EP events based on the one or more extracranial data elements 20D. System 100 may thereby infer pretrained ML based EP detection model 120 on the one or more extracranial data elements 20D, to produce a prediction, or notification of detection 120P of occurrence of at least one EP event (e.g., IED) in the subject (e.g., in extracranial signal 20SG), at the corresponding timeframe 110TF. Prediction 120P may include, for example a binary value, where ‘1’ may indicate occurrence of at least one EP event in the subject, at the corresponding timeframe 110TF, and ‘0’ may indicate that no EP event (e.g., IED) had occurred in the subject, at the corresponding timeframe 110TF.

According to some embodiments, detected EP events may include, for example IED events, pathological events such as sharp waves or subclinical rhythmic epileptiform discharge of adults (SREDA), physiological sleep electrophysiological events such as vertex waves or bouts of theta (4-8 Hz) activities, High frequency oscillation (HFO) events (80-300 Hz), ripple events (80-120 Hz), slow wave (<4 Hz) events, sleep spindle (10-15 Hz) events, and any combination thereof.

According to some embodiments, system 100 may receive, e.g., during a training stage, one or more intracranial EEG signals 30SG, originating from intracranial EEG electrodes 30. System 100 may employ preprocessing module 110 to process intracranial EEG signals 30SG, and produce respective intracranial data elements 30D as elaborated herein (e.g., in a similar manner as extracranial EEG data elements 20D). The one or more intracranial signals 30SG may be substantially concurrent with respective EEG signals 20SG. Therefore, intracranial data elements 30D may be associated with, or correspond to the same timeframes 110TF as intracranial data elements 30D.

As shown in FIG. 2, system 100 may include a second ML based model, denoted herein as intracranial classification model 130 or ML model 130.

According to some embodiments, ML model 130 may be pretrained to receive intracranial EEG data elements 30D, originating from intracranial EEG signals 30SG and automatically produce at least one annotation 130A, indicating occurrence of an EP event (e.g., IED) in the subject at a timeframe 110TF that corresponds to extracranial EEG data 20D of extracranial signal 20SG.

For example, system 100 may receive (e.g., during a training stage) at least one intracranial EEG signal 30SG, originating from at least one respective intracranial EEG electrode 30, and at least one concurrent, training-phase extracranial signal 20SG, from at least one respective extracranial EEG electrode 20.

As elaborated herein, feature extraction module 115 may collaborate with preprocessing module 110 to process the at least one intracranial EEG signal 30SG, so as to obtain one or more respective intracranial data elements 30D. Intracranial data elements 30D may represent electrical activity in the subject's brain, as measured by the at least one intracranial EEG electrode 30, in a timeframe 110TF that is substantially concurrent with at least one respective training-phase extracranial signal 20SG (or corresponding extracranial data element 20D).

Additionally, or alternatively, ML model 130 may receive (e.g., via input 7 of FIG. 1) a corresponding expert annotation data element 40 (e.g., provided by an expert physician), also referred to herein as an EP label data element 40. EP label data element 40 may indicate occurrence of an EP event in the subject (e.g., in intracranial EEG signal 30SG).

For example, EP label data element 40 may include a binary value, where ‘1’ may indicate occurrence of at least one EP event in the subject, at timeframe 110TF, and ‘0’ may indicate that no EP event had occurred in the subject, at timeframe 110TF.

In another example, EP label data element 40 may include annotation of a type of specific occurrence in the subject's brain. For example, EP label data element 40 may include an identification a specific EP event as an IED event, a specific pathological event, a physiological sleep event or stage, an HFO event, a ripple event, a slow wave event, a spindle event, and the like.

System 100 may use EP label data element 40 as supervisory information to pretrain intracranial classification model 130, so as to automatically produce (e.g., without further intervention) at least one automated annotation 130A, based on incoming intracranial data elements 30D of intracranial EEG signals 30SG. Automated annotations 130A may indicate occurrence of EP events in the concurrent, training-phase extracranial signal 20SG of the subject.

System 100 may subsequently use the at least one automated annotation 130A data elements as supervisory information for training EP detection model 120 to produce EP prediction 120P, e.g., to detect occurrence of EP events based on the one or more extracranial data elements.

In other words, ML-based intracranial classification model 130 may be trained based on intracranial EEG signals 30SG, which are difficult to obtain, but are expected to indicate EP event (e.g., IED) occurrence with improved signal to noise ratio (SNR) in relation to EEG signals 20SG originating from non-invasively located electrodes 20. Therefore, supervised training of intracranial classification model 130, based on intracranial EEG signals 30SG may produce automated annotations 130A of EP occurrence events, and automated annotations 130A may in turn be utilized as supervisory information to train EP detection model 120 to predict EP (e.g., IED) occurrence based on easily obtainable, extracranial data elements 20D of non-invasive EEG signals 20SG.

According to some embodiments, system 100 may further include at least one condition categorization module 140 (or categorization module 140, for short). Categorization module 140 may, for example be a rule-based module, configured to receive the predicted, or identified events of EP (e.g., IED) occurrence 120P, and provide a notification 140N (e.g., recommendation, diagnosis, prognosis of a medical condition of the subject), based on the predicted occurrence 120P of EP (e.g., IED) events.

For example, categorization module 140 may calculate one or more EP property data elements 140P, representing statistical characteristics of the detected occurrence of EP events. EP property data elements 140P may include, for example a frequency of identified EP events 120P, a number of identified EP events 120P within predetermined timeframe, a correlation of identified EP events 120P to sleep stages (e.g., frequency of identified EP events 120P within an REM sleep stage), a time (e.g., from commencing sleep) of occurrence of EP events 120P, time-wise distribution or regularity of identified EP events 120P, statistics (e.g., mean, variance) of duration of gaps between consecutive identified EP events 120P, and the like.

Categorization module 140 may subsequently apply rule-based logic on the one or more EP property data elements 140P, to categorize a medical condition of the subject. For example, categorization module 140 may identify a specific subject, as having a property data elements 140P (e.g., frequency of identified EP events 120P) that exceeds a predetermined threshold (e.g., beyond 5 events in an hour), as epileptic, and may subsequently produce a notification 140NR indicating this diagnosis.

In another example, categorization module 140 may be configured to identify a certain number or rate of IEDs following traumatic brain injury or neurosurgery, and may, for example, produce a recommendation notification 140N for a monitoring and follow-up regime that may be more detailed than a default protocol.

Additionally, or alternatively, system 100 may then transmit (e.g., via a communication system such as the Internet) notification 140N (e.g., recommendation, diagnosis, and/or prognosis) to a computing device (e.g., computing device 1 of FIG. 1) of a predefined account (e.g., email account) of a predetermined user (e.g., a caregiver or physician).

Additionally, or alternatively, categorization module 140 may be, or may include a ML-based categorization model 140, that may be pretrained to categorize a medical condition of the subject.

For example, system 100 may receive (e.g., via input 7 of FIG. 1, during a training stage) a condition label data element 40CND, indicating a medical condition of the subject. Condition label 40CND may include, for example, an expert's (e.g., a physician's) annotation representing ground truth of a condition or diagnosis of the subject. System 100 may subsequently use condition label data element 40CND as supervisory information to train categorization model 140 to automatically categorize a medical condition, based on the one or more EP property data elements.

System 100 may subsequently (e.g., during an inference stage), infer pretrained ML-based categorization model 140 on the one or more EP property data elements 140P, to categorize the medical condition of the subject.

For example, notification 140N (e.g., diagnosis, prognosis) of categorization module 140 may relate to medical conditions such as epilepsy, autism, Alzheimer's disease, neurodegeneration, dementia, Traumatic Brain Injury (TBI), Post Traumatic Stress Disorder (PTSD), abnormal brain activity following neurosurgery, existence of brain tumors, anxiety, depression, psychosis, chronic headache or migraine, Attention Deficit Hyperactivity Disorder (ADHD), and stroke. In the above medical conditions, identification of a certain number or rate of IEDs may join other observations in determining clinical diagnosis.

In another example, notification 140N of categorization module 140 may include a recommendation for treatment, or prescription of medication for at least one of the aforementioned medical conditions. For example, identification of a certain number or rate of IEDs following traumatic brain injury or neurosurgery may, in some instances, lead to administration of anti-epileptic drugs as prophylactic measure before seizures occur.

According to some embodiments, the at least one extracranial EEG electrode 20 may include a plurality of extracranial EEG electrodes 20, arranged upon a pad 20P. Pad 20P may, for example, have an adhesive surface adapted to be applied, or fitted to the subject's face, e.g., substantially over the subject's zygomatic bone or maxilla.

According to some embodiments, and as shown in FIG. 2, system 100 may facilitate a feedback mechanism, directing information from categorization module 140 back to preprocessing module 110 and/or feature extraction module 115.

This feedback may, for example, allow system 100 to optimally select specific extracranial EEG electrodes 20 of pad 20P. Additionally, or alternatively, the feedback mechanism may allow fine-tuning of parameters of preprocessing module 110 and/or feature extraction module 115 to improve detection of EP events in extracranial signals 20SG and/or categorization 140N of the subject's condition.

For example, system 100 may obtain extracranial signals 20SG from specific, respective extracranial EEG electrodes 20 by selecting the specific extracranial EEG electrodes 20 among the plurality of extracranial EEG electrodes in pad 20, based on the categorization of the medical condition.

In other words, system 100 may receive a plurality of extracranial signals 20SG from a respective plurality of extracranial EEG electrodes 20. Categorization module 140 may assess the extracranial data elements 20D of each extracranial signal 20SG, to identify one or more (e.g., a subset) of the extracranial signals as most prominent for categorizing the medical condition 140N, according to a predetermined metric (e.g., confidence level, accuracy, and the like). Categorization module 140 may then select at least one extracranial EEG electrode 20 of the plurality of extracranial EEG electrodes that corresponds to, or belongs to the identified subset of extracranial signals.

In another example, system 100 may receive a plurality of extracranial signals 20SG from extracranial EEG electrodes 20 at a respective plurality of positions. Categorization module 140 may assess the extracranial data elements 20D of each extracranial signal 20SG, to identify one or more (e.g., a subset) of the extracranial signals as most prominent for categorizing the medical condition 140N, according to the predetermined metric. Categorization module 140 may then select at least one position of an extracranial EEG electrode 20 that corresponds to, or belongs to the identified subset of extracranial signals.

In such embodiments, system 100 may be applied to predefine optimal locations for placing extracranial EEG electrodes 20, for specific condition categories and subject profiles. For example, given a sufficiently large cohort of subjects, each with their own profile (e.g., gender, age, medical history and suspected diagnosis), system 100 may be applied to provide a notification 140N that would include an optical position for placing, or selecting at least one extracranial EEG electrode 20. The term “optimal” may be used in this context to indicate a location, or selection that is expected to provide maximal performance (e.g., accuracy, confidence level) in identifying EP events, and/or categorizing a condition of the subject.

In another example, the feedback of patient condition categorization 140 may serve to fine-tune a functionality of preprocessing module 110, thereby improving the detection of EP events 120P by detection model 120.

For example, categorization module 140 may (e.g., during a training period of EP detection model 120) perturbate, or change a value of at least one parameter (e.g., of filter 110FLT), based on said categorization of the medical condition. For example, categorization module 140 may collaborate with preprocessing module 110 to change at least one parameter of filter 110FLT, such as a band-pass frequency of 110FLT, a band-stop frequency of 110FLT, an amplification of 110FLT, and the like. Preprocessing module 110 may applying the filter with the at least one changed parameter value on the at least one extracranial signal, to change one or more extracranial data elements 20D. It may be appreciated that the output of EP module 120 (detection of EP events 120P), and consequently change the output of categorization module 140 (categorization of the patient condition) may also be changed, subject to the perturbation of filter 110FLT parameters. These changes may proceed until predetermined thresholds of performance metrics (e.g., accuracy, recall, confidence level, etc.) of EP detection model 120 and/or categorization module 140 are reached.

In another example, the feedback of patient condition categorization 140 may serve to fine-tune a functionality of feature extraction module 110, thereby improving the detection of EP events 120P by detection model 120.

For example, categorization module 140 may (e.g., during a training period of EP detection model 120) change, or perturbate a selection specific features by feature extraction module 115, based on said categorization of the medical condition. It may be appreciated that such perturbation or change in feature extraction module 115 may produce a corresponding change in EEG data elements 20D, and may induce a change in the output of ep module 120 (detection of ep events 120P). The change in the output of ep module 120 may consequently change the output of categorization module 140 (categorization of the patient condition). System 100 may proceed to perform these perturbations until a predetermined thresholds of performance metrics (e.g., accuracy, recall, confidence level, etc.) Of ep detection model 120 and/or categorization module 140 are reached.

The feedback of subject categorization to EEG electrodes 20, preprocessing module 110, and/or feature extraction module 115 may therefore serve to continuously (e.g., repeatedly, over time), and dynamically (e.g., in view of different subjects, conditions, and electrode characteristics) improve the detection of ep events 120P and/or the categorization 140n of subject condition.

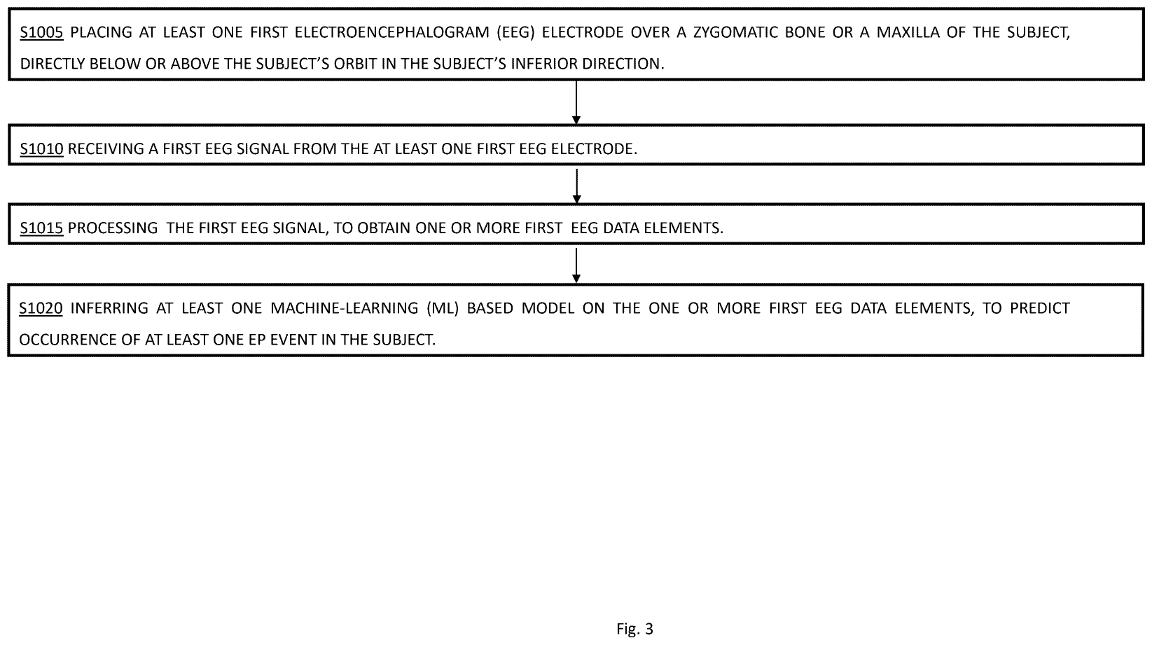

Reference is now made to FIG. 3, which is a flow diagram, depicting a method of detecting ep events in a human subject, according to some embodiments.

As shown in step S1005, embodiments of the method may include placing at least one first EEG electrode 20 over a zygomatic bone, or a maxilla of the subject, directly below or above the subject's orbit in the subject's inferior direction.

As shown in step S1010, embodiments of the method may include receiving, by at least one processor (e.g., processor 2 of FIG. 1), a first EEG signal 20SG from the at least one first EEG electrode.

As shown in step S1015, the at least one processor may process the first EEG signal 20SG, to obtain one or more first EEG data elements 20D.

As shown in step S1020, the at least one processor may subsequently infer at least one ML based model 120 on the one or more first EEG data elements 20D, to predict occurrence of at least one ep event in the subject.

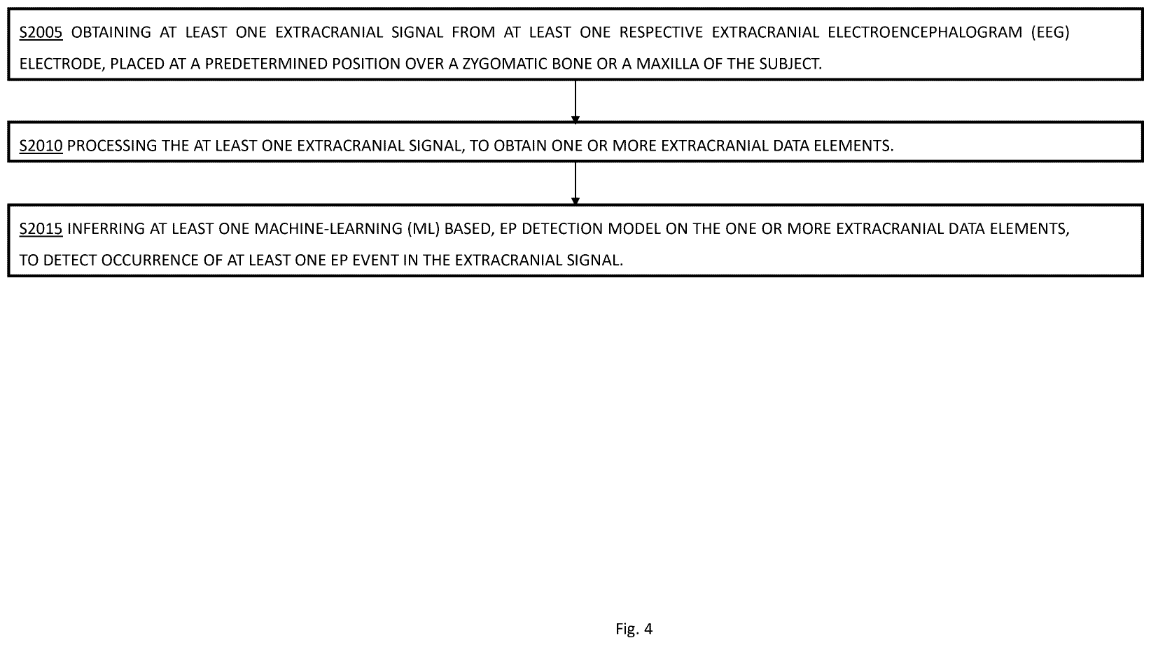

Reference is now made to FIG. 4, which is a flow diagram, depicting another method of detecting EP events in a human subject, by at least one processor (e.g., processor 2 of FIG. 1), according to some embodiments.

As shown in step S2005, the at least one processor may obtain at least one extracranial signal 20SG from at least one respective extracranial EEG electrode 20, placed at a predetermined position over a zygomatic bone or a maxilla of the subject.

As shown in step S2010, the at least one processor may process, or employ signal processing circuitry to process the at least one extracranial signal 20SG, thereby obtaining one or more extracranial data elements 20D.

As shown in step S2015, the at least one processor may subsequently infer at least one ML based, EP detection model 120 on the one or more extracranial data elements, to detect occurrence of at least one EP event in the extracranial signal 20SG.

Depth model: The inventors have configured a first ML based model, denoted herein as “intracranial classifier model 130” or the “depth model” interchangeably, to determine occurrence of EP events based on intracranial EEG electrodes.

During experimentation, a subset of 6 patients and channels were selected for neurologist assessment: three most medial anterior hippocampus, amygdala, and entorhinal cortex (except one patient who hasn't implanted in the entorhinal cortex), all bilateral and re-referenced offline to the average of the earlobe channels, and bipolar reference separately. This montage was cropped and presented for 15 min during the first NREM sleep stage; a few minutes after the patient fell asleep. Then, an expert neurologist, blinded to the patient clinical profile, tagged 337 interictal activity epochs (on average 56 for each patient) using an appropriate software. Each IED tag was annotated on a specific timestamp and specifies the lateralization of the abnormal activity.

The EEG signal was preprocessed by a preprocessing module (denoted herein as preprocessing module 110). During this preprocessing, the EEG signal was resampled to a sample rate of 1 KH and was band-pass filtered digitally between 0.1 Hz and 500 Hz. An additional notch filter at 50 Hz was further applied to the continuous data offline to remove residual line noise. The filters were applied using a Kaiser-type Finite Impulse Response (FIR) filter with zero phase shift. The inventors applied z-score normalization on the raw channels. The continuously tagged data of each patient was segmented into 250 ms epochs and marked either as abnormal or as normal, based on the manual marking. From each epoch, the inventors extracted 25 features (denoted herein EEG data 30D) that represent the statistical and spectral properties of the signal in the current epoch.

The inventors split the data randomly into train-test datasets according to a 75%-25% ratio and kept the inner ratio between normal and abnormal epochs. The inventors trained two tree-based intracranial classifier models 130: random forest (RF) and light gradient boost machine (LGBM), and used the trained models 130 to classify the test dataset. The inventors evaluated the results using a few metrics and methods of estimating machine learning models, starting from K-fold cross-validation. The inventors calculated precision and recall (also called sensitivity) metrics with stratified K-fold cross-validation. The inventors shuffled and split the data randomly into four unlapping folds when each time another fold was used as the test set, and all the other epochs were used as the training set. Each time, the inventors trained and tested the machine, extracted a confusion matrix, calculated precision and recall metrics, and set the average of all folds together as the final result. In addition, the inventors generated a precision-recall curve which is a useful technique to evaluate model performance when the dataset is imbalanced while the common ROC AUC is too optimistic. Additionally, the feature importance of the models was extracted.

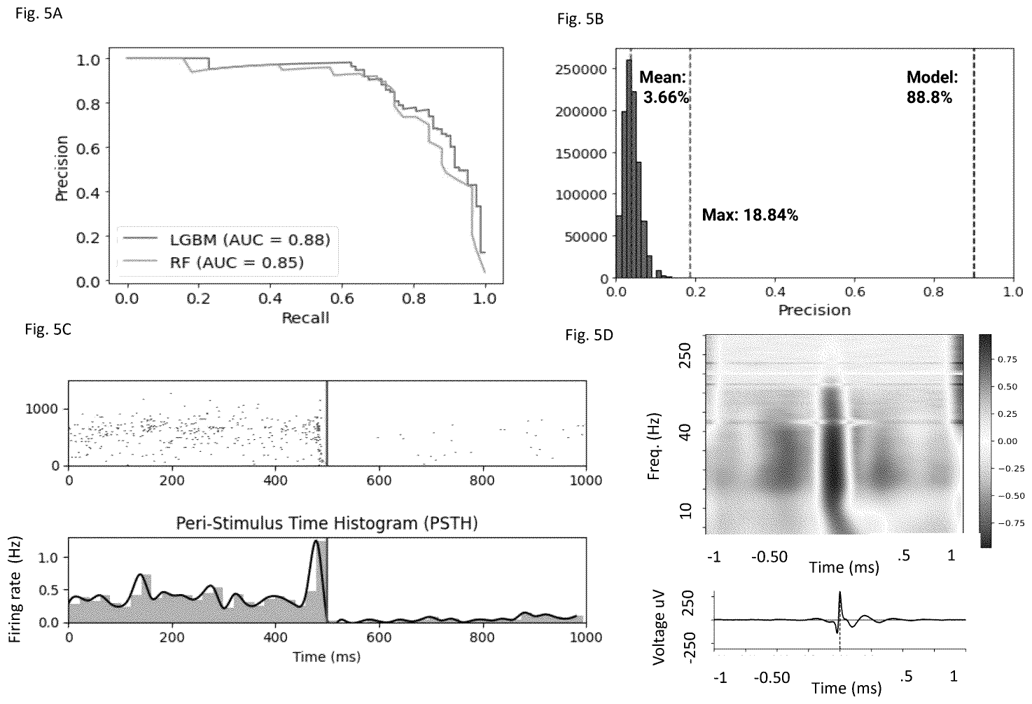

Reference is now made to FIGS. 5A-5D, which show experimental results of detecting EP events in a human subject, by depth model 130, according to some embodiments of the invention.

Precision-recall curves and Area Under the Curve (AUC) for each model represent the tradeoff between these metrics (FIG. 5A). Shuffling test results show a distribution of precision values where the mean precision is marked by the red dashed line and the max precision is marked by the green dashed line (FIG. 5B). The average deep model 130 performance is marked by the black dashed line showing a significantly higher value (FIG. 5C). Microwire data: neuronal spiking signatures of IEDs in right amygdala electrode, the red line marks the IED peak as detected in RA macro electrode. Top panel of FIG. 5D shows TFR of specific channel detections. Bottom panel of FIG. 5D shows average spike in the time domain.

The inventors have configured a second ML based model, denoted herein as “EP detection model 120” or “zEEG model 120”, or “classifier 120” interchangeably, to determine occurrence of EP events based on non-invasive, extracranial EEG electrodes. This was done in two phases, denoted here as a “basic” version of the non-invasive zEEG (zygomatic EEG) model 120, and an “advanced” version of the non-invasive zEEG model 120.

Basic non-invasive zEEG model: As scalp electrodes contain many artifacts and noise, the inventors reject bad epochs. For this goal, the data was segmented into 5 seconds epochs, and then the inventors discarded epochs according to a fixed threshold of amplitude higher than 500 μv for zEEG. The total rejected time was 99 minutes (on average 16 minutes per patient) which corresponded to 4% of the data. In the next step, using the LGBM classifier, the inventors detected IEDs during the whole night and created a binary vector that will correspond to the features vector as the true label. The classifier 120 results were separated for each hemisphere and a final result vector was combined for both sides. The inventors analyzed the importance of each extracranial channel, to find the most effective combination of extracranial EEG electrodes for the model 120. The zygomatic electrodes were chosen according to this feature engineering process.

The inventors used preprocessing module 110 to resample EEG signals 20SG to a sample rate of 1 KH. zEEG channel signals 20SG were band-pass filtered digitally between 0.1 Hz and 40 Hz, and an additional notch filter at 50 Hz was further applied to the continuous data offline to remove residual line noise. Then, the data of each patient was segmented into 250 ms epochs and every epoch was marked as normal or abnormal according to the deep model 130 classifier result. Each epoch included a matrix of 250 time points over 2 channels—the right zEEG channel, and the left zEEG channel. The next steps were similar to those applied to depth model 130: The inventors used z-score normalization and extracted the same type features 20D as for 30D. The inventors trained model 120 and evaluated the results in the same manner.

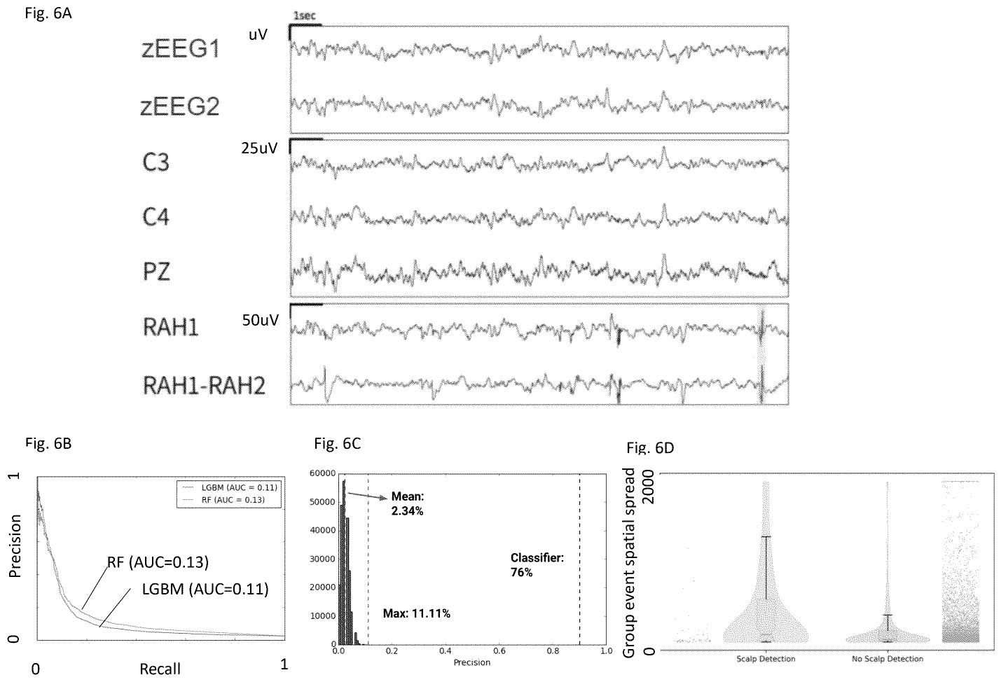

Reference is now made to FIGS. 6A-6D, which show experimental results of detecting EP events in a human subject, by a basic version of a zEEG model 120, according to some embodiments.

FIG. 6A shows an example of MTL IED detection. The blue background marks an epoch that includes IED according to scalp model detection, which was made using only zEEG channels. Note that the scalp channels are flat, making it difficult to distinguish epileptic activity. Some IEDs were missed by the basic zEEG model 120. RAH=right anterior hippocampus. FIG. 6B shows a Precision-Recall curve and AUC of non-invasive mode. FIG. 6C shows shuffling test results compared to the MTL model results. FIG. 6C shows a spatial spread area of IEDs compared between groups of non-invasive detected and undetected events.

Advanced non-invasive zEEG model 120: Using high-density 256-channel EEG signals (covering scalp and facial electrodes), obtained during overnight sleep, the inventors focused on the first NREM epoch for each epilepsy patient and control and cleaned noisy epochs with arousal-related artifacts. The inventors optimized the basic non-invasive zEEG model 120 by testing different filters, electrode choice, referencing methods, ICA preprocessing, as well as augmentation of the training dataset, to produce the advanced version of model 120. The inventors tested EEG channel configuration parameters in each level of implementation and the effect was measured using the ability to differentiate between patients and controls.

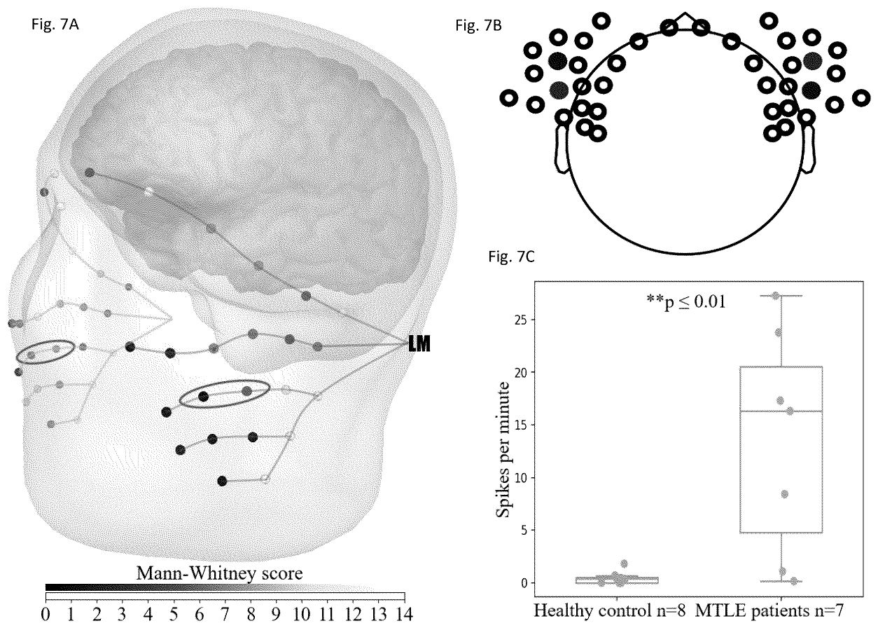

Reference is now made to FIGS. 7A-7C, which show experimental results showing selection of EEG electrodes by an advanced version of a zEEG model, according to some embodiments of the invention.

FIG. 7A shows three-dimensional (3D) rendering of hd-EEG facial electrodes and the statistical significance associated with their ability to separate MTLE patients vs. healthy controls. (Mann-Whitney score according to the color bar below. Low indicates high significance). Note high significance of zEEG electrodes around cheeks/below eye orbits. Red ellipse marks pairs of zEEG electrodes used for subsequent quantification FIG. 7B shows a top view of facial electrodes in the hd-EEG montage. Red and blue electrodes mark bilateral pairs of zEEG electrodes used for non-invasive detection of MTL IEDs during sleep. FIG. 7C shows a box-plot histogram depicts the difference in rate of IED (events per minute) between healthy control group (green, left) and MTLE patients (orange, right).

In the first level, the depth model 130, the inventors ran the following options: for the algorithm the inventors tested Random Forest, LGBM and XGB classifiers, for the number of channels that were used, the inventors tested the deepest electrode, 2 deepest or 3 deepest, for the brain area the inventors tested only the hippocampus or the whole MTL including amygdala, entorhinal cortex and para-hippocampal gyrus, and for the reference method the inventors used bipolar reference, Cz reference or both.

In the second level, basic zEEG model 120, the inventors ran the following options: for the algorithm the inventors tested Random Forest, LGBM and XGB classifiers, for the area the inventors used electrodes on the scalp or on the cheeks, for the filters the inventors used low-pass filter of 40 Hz or 70 Hz, for the ratio of spikes and non-spikes epochs in the training dataset the inventors used the raw data, ratio of 1-10 and balanced dataset, for the symmetry of the dataset the inventors used the raw data or a “doubled” dataset in which the laterality was balanced, for the confidence level the inventors used the whole range between confidence ratings of 50-99.

In the third level, the preprocessing of hd-EEG of the advanced model 120, the inventors ran the following options: for the reference the inventors used Cz, bipolar, and average of right and left mastoid, for the filters the inventors used high-pass filter of 0.1 Hz or 0.3 Hz and low-pass filter of 40 Hz or 70 Hz, for location of electrodes the inventors used Cz, each combination of two electrodes on the face, or average of 4-6 of them, for cardio artifacts the inventors cleaned the signal using ICA, and for the epochs that were chosen for analysis the inventors used the first episode of NREM sleep or the entire REM sleep data.

This fine-tuning gave rise to an advanced non-invasive EEG model 120 that revealed, among the 40 facial electrodes, the topography of the optimal area for zEEG sensors that is most sensitive to IED detection and yields the best separation between MTLE patients and healthy controls. The final model was also run on a separate validation dataset of patients and controls, without any fine-tuning or modifications, thereby ensuring the model's validity and robustness in avoiding overfitting and generalizing beyond the initial group of patients and controls. This model's result enabled the diagnosis of epileptic activity during sleep according to a certain threshold that was calculated according to the training and testing dataset. At present, a rate of more than five detected events suspected as interictal spikes per minute of NREM sleep seems best to reliably differentiate MTLE patients from controls.

Analysis of LFP and neuronal spiking activities in microwire data: Neuronal clusters were identified using appropriate software: extracellular recordings were high-pass filtered above 300 Hz and a threshold of 5 standard deviations above the median noise level was computed. Detected events were clustered (or categorized as noise) using automatic superparamagnetic clustering of wavelet coefficients, followed by manual refinement based on the consistency of spike waveforms and inter-spike interval distributions.

Trials of IEDs were aligned according to the closest macro electrode detection.

IEDs characteristics and detection correlations: In order to understand which IEDs were better detected, the inventors analyzed IEDs features in different aspects, including for example: morphology (amplitude, gradient, duration), spectral properties (power in different bands), or spatial dimension (propagation speed and area).

For investigating spatial propagation, the inventors used the anatomical coordinates based on CT and MRI imaging data.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of detecting Electrophysiological (EP) events in a human subject by at least one processor, the method comprising:
obtaining at least one extracranial signal from at least one respective extracranial electroencephalogram (EEG) electrode, placed at a predetermined position over a zygomatic bone or a maxilla of the subject;
processing the at least one extracranial signal, to obtain one or more extracranial data elements; and
inferring at least one machine-learning (ML) based, EP detection model on the one or more extracranial data elements, to detect occurrence of at least one EP event in the extracranial signal.

2. The method of claim 1, wherein the predetermined position is directly below the subject's orbit in the subject's inferior direction.

3. The method of claims 1, wherein the EP events are selected from a list consisting of Interictal Epileptic Discharges (IEDs), pathological events, physiological sleep electrophysiological events, High Frequency Oscillation (HFO) events, ripple events, slow wave events, and spindle events.

4. The method of claim 1, wherein the at least one ML based detection model is a decision-tree based model, selected from a random forest model, a Light Gradient Boost Machine (LGBM) model, a gradient-boost ML model, and an Extreme Gradient Boost (XGB) model.

5. The method of claim 1, wherein the at least one EP detection model is pretrained to detect the occurrence of EP events based on the one or more extracranial data elements.

6. The method of claim 4, wherein training the EP detection model comprises:
obtaining an ML-based, intracranial classification model that is pretrained to automatically produce at least one annotation indicative of occurrence of EP in the subject; and
using the automatically produced annotation as supervisory data, to train the EP detection model, so as to detect occurrence of EP events based on the one or more extracranial data elements.

7. The method of claim 4, wherein training the intracranial classification model comprises:
receiving at least one intracranial EEG signal, originating from at least one respective intracranial EEG electrode;
receiving at least one concurrent, training-phase extracranial signal from at least one respective extracranial EEG electrode;
processing the at least one intracranial EEG signal, to obtain one or more respective intracranial data elements;
receiving an EP label data element, indicating occurrence of an EP event in the subject; and
using the EP label data element as supervisory information to train the intracranial classification model to produce the at least one annotation, based on the one or more intracranial data elements, wherein said annotation indicates occurrence of EP events in the concurrent, training-phase extracranial signal.

8. The method of claim 1 further comprising:
calculating one or more EP property data elements, representing statistical characteristics of the detected occurrence of EP events; and
applying rule-based logic, or a pretrained ML-based categorization model on the one or more EP property data elements, to categorize a medical condition of the subject.

9. (canceled)

10. The method of claim 8, wherein training the categorization model comprises:
receiving a condition label data element, indicating a medical condition of the subject; and
using the condition label data element as supervisory information to train the categorization model to automatically categorize a medical condition, based on the one or more EP property data elements.

11. The method of claim 8 wherein obtaining the extracranial signal from at least one respective extracranial EEG electrode comprises selecting the at least one extracranial EEG electrode among a plurality of extracranial EEG electrodes, based on said categorization of the medical condition.

12. The method of claim 8 wherein obtaining the extracranial signal from at least one respective extracranial EEG electrode comprises:
receiving a plurality of extracranial signals from a respective plurality of extracranial EEG electrodes;
identifying a subset of the extracranial signals as most prominent for categorizing the medical condition; and
selecting at least one extracranial EEG electrode of the plurality of extracranial EEG electrodes that corresponds to said subset of extracranial signals.

13. The method of claim 11, wherein the plurality of extracranial EEG electrodes are arranged upon a pad, adapted to be applied to the subject's face, substantially over the zygomatic bone or a maxilla of the subject.

14. The method of claim 8 wherein obtaining the extracranial signal from at least one respective extracranial EEG electrode comprises:
receiving a plurality of extracranial signals from extracranial EEG electrodes at a respective plurality of positions;
identifying a subset of the extracranial signals as most prominent for categorizing the medical condition; and
determining a position of at least one extracranial EEG electrode based on the identified subset of extracranial signals.

15. The method of claim 8 wherein processing the at least one extracranial signal comprises:
determining a value of at least one parameter of a filter, based on said categorization of the medical condition; and
applying the filter with the at least one parameter value on the at least one extracranial signal, to obtain the one or more extracranial data elements.

16. The method of claim 8, wherein said medical condition is selected from a list consisting of Epilepsy, Autism, Alzheimer's disease, Neurodegeneration, dementia, Traumatic Brain Injury (TBI), Post Traumatic Stress Disorder (PTSD), abnormal brain activity following neurosurgery, existence of brain tumors, anxiety, depression, psychosis, chronic headache or migraine, Attention Deficit Hyperactivity Disorder (ADHD) and stroke.

17. A system for detecting EP events in a human subject, the system comprising:
an EEG device, coupled with one or more EEG electrodes;
a non-transitory memory device, wherein modules of instruction code are stored; and
at least one processor associated with the memory device, and configured to execute the modules of instruction code,
whereupon execution of said modules of instruction code, the at least one processor is configured to:
obtain, via the EEG device at least one extracranial signal from at least one respective extracranial EEG electrode, placed at a predetermined position over a zygomatic bone or a maxilla of the subject, directly below the subject's orbit in the subject's inferior direction;
process the at least one extracranial signal, to obtain one or more extracranial data elements; and
infer at least one ML based, EP detection model on the one or more extracranial data elements, to detect occurrence of at least one EP event in the extracranial signal.

18. (canceled)

19. (canceled)

20. (canceled)

21. (canceled)

22. The system of claim 17, wherein the at least one processor is configured to train the EP detection model by:
obtaining an ML-based, intracranial classification model that is pretrained to automatically produce at least one annotation indicative of occurrence of EP in the subject; and
using the automatically produced annotation as supervisory data, to train the EP detection model, so as to detect occurrence of EP events based on the one or more extracranial data elements.

23. The system of claim 17, wherein the at least one processor is configured to train the intracranial classification model by:
receiving at least one intracranial EEG signal, originating from at least one respective intracranial EEG electrode;
receiving at least one concurrent, training-phase extracranial signal from at least one respective extracranial EEG electrode;
processing the at least one intracranial EEG signal, to obtain one or more respective intracranial data elements;
receiving an EP label data element, indicating occurrence of an EP event in the subject; and
using the EP label data element as supervisory information to train the intracranial classification model to produce the at least one annotation, based on the one or more intracranial data elements, wherein said annotation indicates occurrence of EP events in the concurrent, training-phase extracranial signal.

24. The system of claim 17 wherein the at least one processor is further configured to:
calculate one or more EP property data elements, representing statistical characteristics of the detected occurrence of EP events; and
apply rule-based logic, or a pretrained ML-based categorization model on the one or more EP property data elements, to categorize a medical condition of the subject.

25. (canceled)

26. (canceled)

27. (canceled)

28. The system of claim 24 wherein the at least one processor is configured to obtain the extracranial signal from at least one respective extracranial EEG electrode by:
receiving a plurality of extracranial signals from a respective plurality of extracranial EEG electrodes;
identifying a subset of the extracranial signals as most prominent for categorizing the medical condition; and
selecting at least one extracranial EEG electrode of the plurality of extracranial EEG electrodes that corresponds to said subset of extracranial signals.

29. (canceled)

30. (canceled)

31. (canceled)

32. (canceled)
